(19) United States
(12) Patent Application Publication
(10) Pub. No.: US 2009/0131943 A1
(43) Pub. Date: May 21, 2009

(54) MEDICAL INSTRUMENT FOR DISTRACTION OR COMPRESSION

(76) Inventors: Craig L. Fischer, Arnold, MO (US); Greg G. Utt, Warrenton, MO (US)

Correspondence Address: GRACE J FISHEL, 2200 WEST PORT PLAZA DRIVE, SUITE 202, ST. LOUIS, MO, 63146, US

(21) Appl. No.: 12/267,022

(22) Filed: Nov. 7, 2008

Related U.S. Application Data
(60) Provisional application No. 60/986,337, filed on Nov. 8, 2007.

(51) Int. Cl. A61B 17/58

(52) U.S. Cl. 606/90

(57) ABSTRACT

A medical instrument for use in orthopedic distraction or compression. The instrument has a pair of handles and a pair of blades removably attached to an actuator. The handles and the blades may be attached on either side of the actuator such that the blades may be used on the left or right side of the spine or other structure being distracted or compressed and such that the handles may be removed thus freeing up space in the incision or portal for other instruments. The handles and the blades are connected to the actuator through a ratchet and pawl mechanism whereby pressing the handles together causes the blades to be spread apart at their distal end while maintaining a window between the blades at their proximal ends to optimize visualization of the operative site. Movement of the blades at their proximal ends is amplified at their distal ends.

This application claims priority from provisional application 60/986,337, filed Nov. 8, 2007, for Medical Instrument for Distraction or Compression.

### BACKGROUND OF THE INVENTION

1. Field of the Invention

The present invention relates to a medical instrument for use in orthopedic distraction or compression.

2. Brief Description of the Prior Art

Various devices exist for distracting or compressing tissues such as bones (e.g., vertebrae). In these devices, most commonly, a pair of blades are mounted at a right angle on a rack or on a threaded rod. One of the blades is fixed to the rack or the rod and the other of the blades is attached to a body that is moved along the rack or rod with a pinion or a worm gear. As the blades are brought together in distraction or compression, the viewing window between the blades is narrowed thus interfering with visualization of the operative site. Commonly, also, existing devices are designed for use on the right or left side of the spine, for example, such that it is necessary to inventory both a right and left-handed device.

In view of the above, it is seen that prior distraction or compression devices are not always useful, or may be awkward to use, in certain surgical situations.

### BRIEF SUMMARY OF THE INVENTION

In view of the above, it is an object of the present invention to provide an improved medical instrument for distraction or compression. It is another object to provide an instrument wherein the viewing window between the proximal end of the blades remains open. It is also an object to provide an instrument that can be used on the right or left side of a spine. Other objects and features of the invention will be in part apparent and in part pointed out hereinafter.

In accordance with the invention, a medical instrument and an actuator for distraction or compression is provided. The actuator has first and second spaced apart shafts, at least one of which is rotatable in the actuator and at least one of which has a ratchet. The other of the shafts has a pawl which is spring biased into engagement with the ratchet. Fittings are provided on the actuator for removable attachment of a pair of blades and a pair of handles on opposite sides and at substantially a right angle. The blades and handles are adapted for universal attachment to the fittings such that the handles and blades may be interchanged on the actuator for right and left-handed use. The ratchet and the pawl are configured such that pressing the handles together causes the blades to move apart in distraction or, if the blades cross over, move together in compression. A push button release is provided for disengaging the pawl from spring biased engagement with the ratchet. The shafts of the actuator are spaced apart such that there is there is viewing window between the proximal end of the attached blades.

The invention summarized above comprises the constructions hereinafter described, the scope of the invention being indicated by the subjoined claims.

### BRIEF DESCRIPTION OF THE SEVERAL VIEWS OF THE DRAWING

In the accompanying drawings, in which several of various possible embodiments of the invention are illustrated, corresponding reference characters refer to corresponding parts throughout the several views of the drawings in which:

FIG. 1 is a photograph of a medical instrument for distraction or compression illustrated in connection with a spine and with the handles removed;

FIG. 2 is a top plan view of an actuator with blades attached illustrated in fully closed position;

FIG. 3 is a top plan view of the actuator in distraction position;

FIG. 4 is a front elevation of the actuator with blades attached;

FIG. 5 is a rear elevation of actuator with the handles and blades attached;

FIG. 6 is a top plan view of the actuator with blades in fully closed position;

FIG. 6A is a cross-section taken along the plane of A-A in FIG. 6;

FIG. 6B is a cross-section taken along the plane of B-B in FIG. 6;

FIG. 7 is a top plan view of a second actuator with blades in fully closed position;

FIG. 7A is a cross-section taken along the plane of A-A in FIG. 7; and,

FIG. 7B is a cross-section taken along the plane of B-B in FIG. 7.

### DETAILED DESCRIPTION OF THE INVENTION

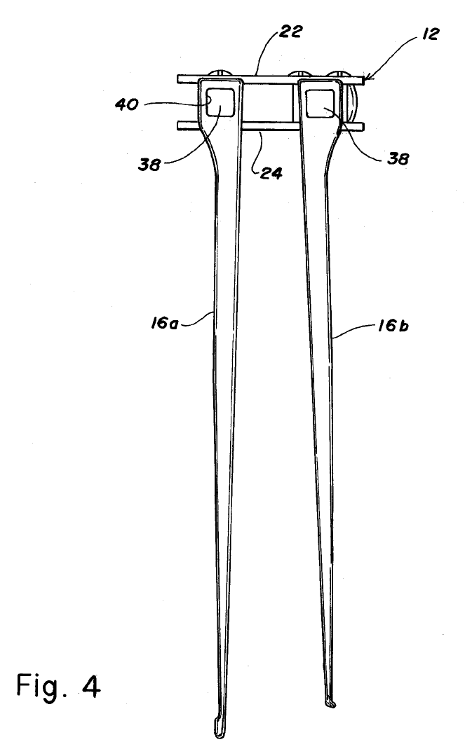

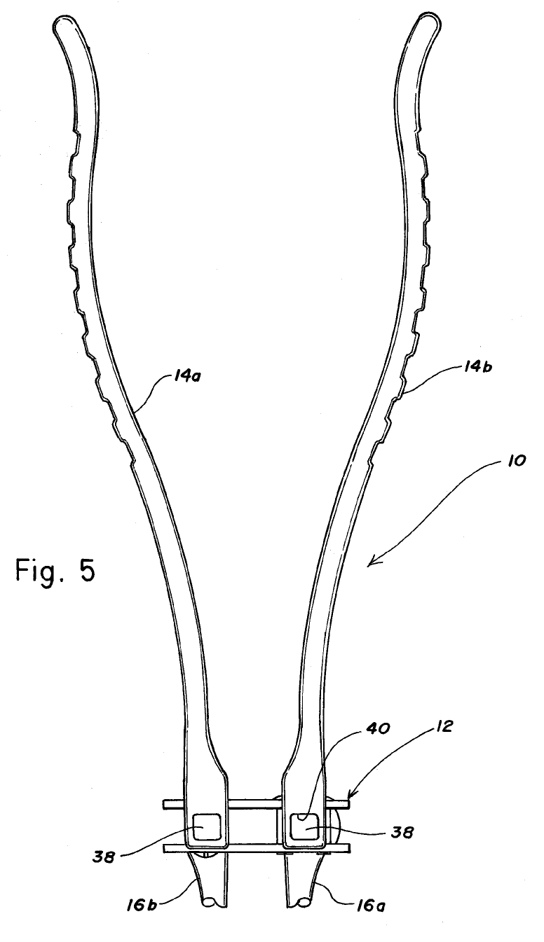

Referring to the drawings more particularly by reference character, reference numeral 10 refers to a medical instrument in accordance with the present invention. In major part, instrument 10 includes an actuator 12, first and second handles 14a, 14b, respectively (FIG. 5), and first and second blades 16a, 16b, respectively (FIG. 4). Handles 14a, 14b and blades 16a, 16b are removably attached substantially at a right angle to actuator 12 and on opposite sides thereof. Blades 16a, 16b and handles 14a, 14b may take various forms. For example, blades 16a, 16b may vary in length and width, be blunt or pointed, straight or curved, etc. as will occur to those skilled in the art, the present invention not being limited to a particular type of blade, including blades that cross over for use in compression. In similar manner handles 14a, 14b may include finger loops, etc.

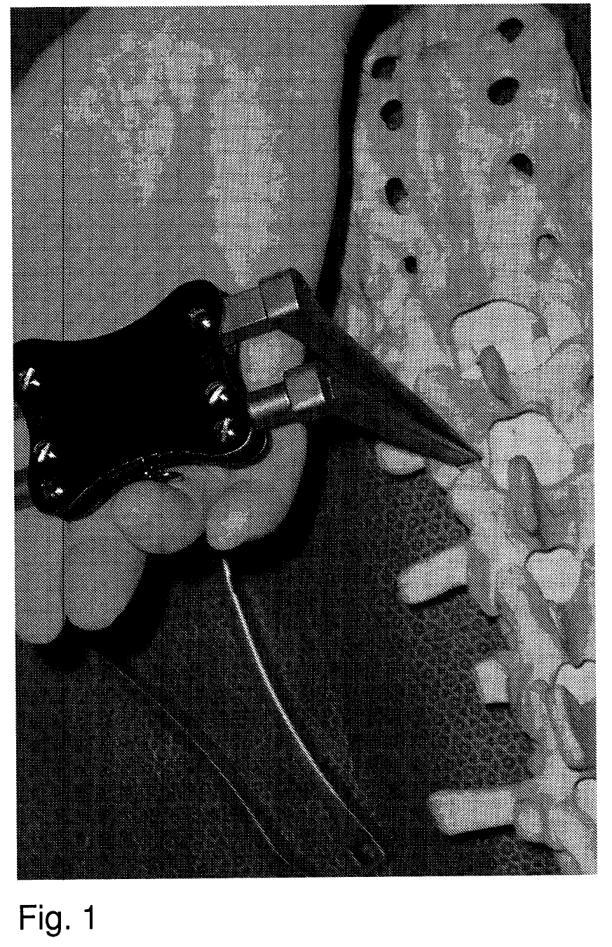

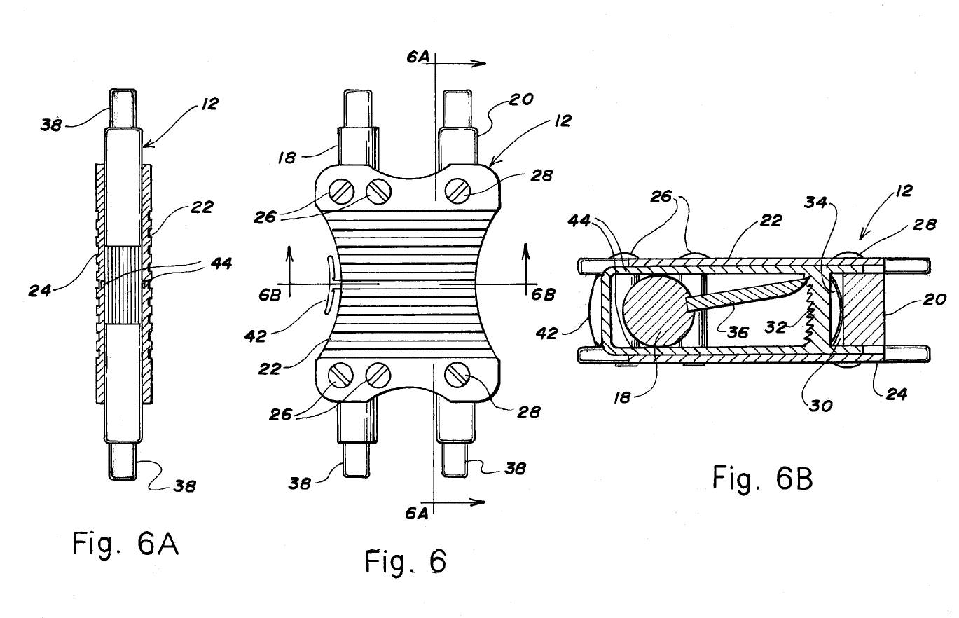

Actuator 12 includes first and second spaced apart parallel shafts 18, 20, respectively, which are sandwiched between upper and lower plates 22, 24, respectively. At least one of shafts 18, 20 is rotatable and the other of which may be fixed between plates 22, 24. As illustrated in FIGS. 1-6 first shaft 18 is a round rod and rotatable and second shaft 20 is square and fixed. As best seen in FIG. 6B, a pair of screws 26 at each end of actuator 12 join upper and lower plates 22, 24. Screws 26 are received in grooves (not shown) provided in first shaft 18. First shaft 18 rotates on screws 26 and screws 26 prevent first shaft 18 from moving longitudinally between plates 22, 24. Second shaft 20 is fixed between plates 22, 24 with screws 28 and includes a pocket 30 within which a ratchet 32 in the form of a radial gear block is spring biased 34. A pawl 36 is provided on first shaft 18 in engagement with ratchet 32.

First and second shafts 18, 20 are provided with identical fittings 38 and the proximal ends of handles 14a, 14b and blades 16a, 16b are adapted for universal attachment to fittings 38. In the form illustrated, fittings 38 are squared ends of first and second shafts 18, 20 for receipt in squared apertures 40 provided in handles 14a, 14b and blades 16a, 16b. This permits handles 14a, 14b and blades 16a, 16b to be attached to either end of actuator 12 for use on the right or left of a spine, for example. A spring biased detent (not shown) or the like may be provided in blades 16a, 16b such that they do not inadvertently detach from shafts 18, 20. No detent, however, may be provided for handles 14a, 14b so that they may be easily detached during a surgical procedure. As illustrated in FIGS. 1-6, pawl 36 is attached to first shaft 18 and ratchet 32 to second shaft 20 but these elements may be reversed as will occur to one skilled in the art for example by placing ratchet 32 on first shaft 18 and placing a spring biased pawl on second shaft 20.

The ratchet 32 and pawl 36 are configured such that squeezing handles 14a, 14b together causes blades 16a, 16b as shown in FIGS. 1-6 to spread apart in distraction whereas blades that cross over squeeze together in compression. Movement of blades 16a, 16b at their proximal ends is amplified at their distal ends. Once blades 16a, 16b are placed in distraction or compression, handles 14a, 14b may be removed as pawl 36 keeps blades 16a, 16b from being released. When it is desirable that blades 16a, 16b be released, a push button release may be provided. As best seen in FIG. 6B, button 42 is mounted on a pair of legs 44 received in grooves provided in upper and lower plates 22, 24. A free end of legs 44 pushes radial gear block serving as ratchet 32 back in pocket 30 releasing pawl 36 and allowing blades 16a, 16b to relax.

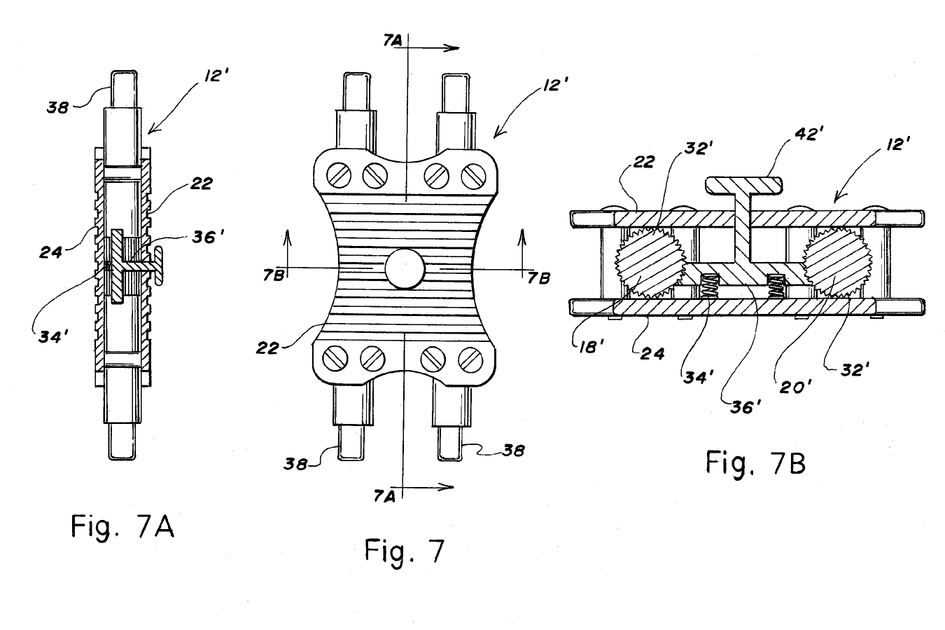

A second embodiment of actuator 12′ is shown in FIG. 7. In this instance, ratchet 32′ is formed in first and second shafts 18′, 20′ both of which rotate in actuator 12′. A double ended pawl 36′ is spring biased 34′ against ratchets 32′. Pressure on button 42′ depresses springs 34′ and releases pawl 36′ from ratchets 32′.

In use, appropriate blades 16a, 16b are selected for the surgical purpose and attached to actuator 12 for use on the right or left side of the tissue to be distracted or compressed. Handles 14a, 14b are attached to the other end of actuator 12.

The purposes to which medical instrument 10 may be used include but are not limited to surgical procedures of the spine such as:

Traditional (“Open”) Procedures

Anterior Cervical Discectomy and Fusion

Anterior Lumbar Interbody Fusion (ALIF)

Cervical Corpectomy

Cervical Foraminotomy

Cervical Laminoplasty

Decompressive Lumbar Laminectomy

Interbody Fusion with Cages (interbody fusion devices)

Posterior Lumbar Interbody Fusion (PLIF)

Spinal Fusion

Posterolateral Gutter Spine Fusion

Transforaminal Lumbar Interbody Fusion (TLIF)

Minimally Invasive Procedures

Vertebroplasty

Kyphoplasty

Microscopic Discectomy

Disc Decompression

Endoscopic Scoliosis Surgery

Minimally Invasive Bone Grafting Procedures

Nucleus Delivery.

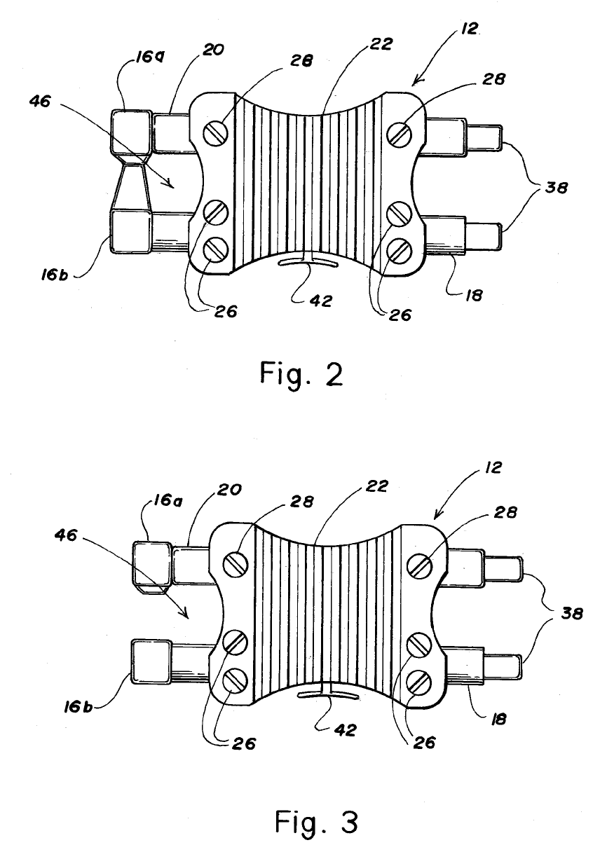

After blades 16a, 16b have been set in distraction or compression, handles 14a, 14b may be removed thus freeing up space in the incision or portal for other instruments. As seen by comparing FIG. 2 with FIG. 3, actuator 12 provides a window 46 between blades 16a, 16b at their proximal end that optimizes visualization of the operative site. Actuator 12 also has a very low profile opening up working space.

In view of the above, it will be seen that the several objects of the invention are achieved and other advantageous results attained. As various changes could be made in the above constructions without departing from the scope of the invention, it is intended that all matter contained in the above description or shown in the accompanying drawings shall be interpreted as illustrative and not in a limiting sense.

What is claimed:

1. A medical instrument for distraction or compression comprising:
an actuator with first and second spaced apart parallel shafts, at least one of said shafts rotatable in the actuator, at least one of said shafts having a ratchet, at least one pawl spring biased in engagement with the at least one ratchet, said first and second shafts having first and second ends,
a first handle removably attached to the first end of the first shaft and a second handle removably attached to the first end of the second shaft, said first and second handles at an angle to the first and second shafts and at least one of said handles rotating the shaft to which it is removably attached,
a first blade removably attached to the second end of the first shaft and a second blade removably attached to the second end of the second shaft, said blades attached at an angle to the first and second shafts and at least one of said blades rotating with the shaft to which it is removably attached, said blades opposite to the handles,
an operator on the actuator for selectively disengaging the at least one spring biased pawl from the at least one ratchet.

2. A medical instrument for distraction or compression comprising:
an actuator with first and second spaced apart parallel shafts, said first shaft rotatable in the actuator with respect to said second shaft, one of said first and second shafts having a ratchet and the other of said first and second shafts having a pawl spring biased in engagement with the ratchet, said first and second shafts having first and second ends,
a first handle removably attached to the first end of the first shaft for rotation of the first shaft and a second handle removably attached to the first end of the second shaft, said first and second handles at an angle to the first and second shafts,
a first blade removably attached to the second end of the first shaft for rotation with the first shaft and a second blade removably attached to the second end of the second shaft, said blades attached at an angle to the first and second shafts and opposite to the handles,
an operator on the actuator for selectively disengaging the spring biased pawl from the ratchet.

3. A medical instrument for distraction or compression comprising:
an actuator with first and second spaced apart parallel shafts, each of said first and second shafts rotatable in the actuator and having opposing ratchets, at least one pawl spring biased in engagement with the opposing ratchets, said first and second shafts having first and second ends,
a first handle removably attached to the first end of the first shaft for rotation of the first shaft and a second handle attached to the first end of the second shaft for rotation of the second shaft, said first and second handles at an angle to the first and second shafts,
a first blade attached to the second end of the first shaft for rotation with the first shaft and a second blade attached to the second end of the second shaft for rotation with the second shaft, said blades attached at an angle to the first and second shafts and opposite to the handles,
an operator on the actuator for selectively disengaging at least one spring biased pawl from the ratchets.

4. An actuator for a medical instrument for distraction or compression, said actuator having first and second spaced apart parallel shafts, at least one of said shafts rotatable in the actuator, at least one of said shafts having a ratchet, at least one pawl spring biased in engagement with the at least one ratchet, said first and second shafts having first and second ends with fittings adapted for universal attachment to first and second handles and first and second blades, said actuator having a push button release of the at least one pawl from spring biased engagement with the at least one ratchet.
